(19) United States
(12) Patent Application Publication
(10) Pub. No.: US 2022/0265716 A1
(43) Pub. Date: Aug. 25, 2022

(54) ANTIBODY PRE-LOADED CD16+NK-92 CELLS AS AN EFFECTIVE THERAPEUTIC PRODUCT FOR TUMOR LYSIS

(71) Applicant: NANTKWEST, INC., San Diego, CA (US)

(72) Inventors: Barry Simon, San Diego, CA (US); Manju Saxena, Culver City, CA (US); Syed Raza Ali, Culver City, CA (US); Joseph Thibault, Culver City, CA (US)

(21) Appl. No.: 17/630,075

(22) PCT Filed: Jul. 27, 2020
(86) PCT No.: PCT/US20/43690

§ 371 Date: Jan. 25, 2022

Related U.S. Application Data
(60) Provisional application No. 62/879,111, filed on Jul. 26, 2019.

(51) Int. Cl. A61K 35/17; A61K 39/395; A61K 45/06; A61P 35/00

(52) U.S. Cl. CPC A61K 35/17; A61K 39/39558; A61K 45/06; A61P 35/00; A61K 2039/545

(57) ABSTRACT

Provided herein are pharmaceutical compositions, comprising a pharmaceutically acceptable carrier and therapeutically effective amounts of high affinity Natural Killer (haNK) cells and a therapeutic antibody in the form of a combined preparation. Also provided herein are methods for treating cancer by using the pharmaceutical composition comprising the haNK cells and the therapeutic antibody.

This application claims priority to our copending U.S. Provisional patent Application with the Ser. No. 62/879,111, which was filed Jul. 26, 2019, and which is incorporated by reference herein.

### FIELD OF THE INVENTION

The present disclosure relates to compositions, kits, and methods for treating cancer, and in particular treating cancer with high affinity natural killer (haNK) cells pre-loaded with a therapeutic antibody.

### BACKGROUND OF THE INVENTION

The background description includes information that may be useful in understanding the present disclosure. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Natural killer (NK) cells are known to play a role in mediating innate immunity, in enhancing adaptive immune responses, and have been implicated in mediating anti-tumor responses via antibody-dependent cell-mediated cytotoxicity (ADCC) by reactivity of CD16 with the Fc region of human IgG1 antibodies. Several NK cell lines are known to have therapeutic effects on cancer patients, such as patients with leukemias and lymphomas. The NK cells may also be further engineered to enhance the cancer cell killing effect—one such technique is the high affinity NK (haNK) cells, which are NK cells that incorporate a high binding affinity receptor that binds to an administered antibody.

Combinations therapies of NK cells and antibodies for the treatment of cancer are known. PCT/US2018/032281 discloses treatment of chordoma by co-administration of an anti-EGFR antibody and high affinity NK cells (haNK). The disclosure provides that the antibody is non-covalently bound to a high affinity variant of a CD16 receptor, or the antibody is administered before transfusion of the haNK cells to so target the chordoma cells for cytotoxic cell killing by the haNK cells.

Most monoclonal antibodies in oncology are administered in body-size-based dosing schedules. This partially controls the variability in both drug distribution and elimination between patients. However, dosing is a challenge in existing cell therapies such as PCT/US2018/032281 where a combination of engineered cells and therapeutic antibodies are administered following different protocols. Further, repeated bolus increases the treatment cost.

Thus, there is a need in the art for new compositions and methods for controlling the viability and efficacy of cell therapeutics, as well as dosing of the engineered cells and therapeutic antibodies. Preferably such as combination would combine both components in one dosing protocol.

### SUMMARY OF THE INVENTION

The inventors have discovered compositions, methods, and devices that enable the generation of a cryopreserved product comprising of CD16⁺NK-92 (haNK) cells pre-loaded with therapeutic antibody. Advantageously and unexpectedly, the compositions disclosed herein remove the increased treatment cost from repeated bolus, and results in an efficacious drug distribution in patients.

In one aspect of the inventive subject matter, the inventors contemplate a pharmaceutical composition comprising a pharmaceutically acceptable carrier and therapeutically effective amounts of high affinity Natural Killer (haNK) cells and a therapeutic antibody in the form of a combined preparation.

Contemplated haNK cells are preferably administered at a dosage of between 5×10⁵ cells/kg and 5×10⁸ cells/kg, and it is further preferred that the haNK cells are a NK92 derivative and/or typically intracellularly express recombinant IL2. Moreover, it is generally preferred that the haNK cell is genetically engineered to have a reduced expression of at least one inhibitory receptor and/or that the haNK cell is genetically engineered to express a CD16 158V variant. Moreover, the haNK cell may be irradiated before administration at a radiation dose of at least 500 cGy.

The antibody contemplated herein may be any therapeutic antibody. For example, the antibody may be anti-VEGF, anti-HER2, anti-EGFR, anti-CTLA4, anti-CD20, anti-CD54, anti-CD33, anti-CD16, and/or anti-CD30 antibody. The antibody may also be selected from the group consisting of Atezolizumab, Ofatumumab, Ipilimumab, Ramucirumab, Olaratumab, Elotuzumab, Necitumumab, Daratumumab, Dinutuximab, Avelumab, Durvalumab, Trastuzumab, Alemtuzumab, Bevacizumab, Pertuzumab, Obinutuzumab, Rituximab, and Cetuximab.

In some preferred embodiments, the haNK cells and the antibody are chemically conjugated, for example by click chemistry.

Moreover, the pharmaceutical composition may further comprise a cryopreservation medium, such as CryoStor CS10. Ideally, the cryopreservation medium is selected such that it favors antibody binding to haNK cells.

Also disclosed herein is a method of making a composition by the steps of (a) mixing haNK cells with a therapeutic antibody to making a combined preparation; (b) freezing the combined preparation; and (c) thawing the combined preparation. In some cases, the haNK cells are in a media comprising about 5% human albumin. Preferably, the haNK cells and therapeutic antibody mixture is mixed with an equivalent volume of a cryopreservation medium. The combined preparation of haNK cells and the therapeutic antibody may be frozen to a temperature less than −80° C., or in some cases to a temperature less than −120° C. The frozen combined preparation is irradiated with a fixed dose of X-ray irradiation to impair the proliferative ability of the haNK cells. In some embodiments, after the thawing step, the combined preparation is incubated on ice or room temperature.

In another aspect, disclosed herein is a method of treating a patient having cancer, comprising administering to the patient a comprising a combined preparation having therapeutically effective amounts of high affinity Natural Killer (haNK) cells and a therapeutic antibody. The composition may be administered intravenously, intratumorally, or by transfusion.

In some cases, the method may also further comprise a step of administering a further cancer treatment to the patient. The further cancer treatment may be an immune therapy, chemotherapy, or radiation therapy. The immune therapy comprises administration of recombinant yeast or recombinant vims expressing a patient- and tumor-specific neoepitope. The chemotherapy may comprise administration of at least one of aldoxorubicin, cyclophosphamide, irinotecan, gemcitabine, capecitabine, 5-FU, FOLFIRL FOLFOX, and oxipiatin. The further cancer treatment is administered separately, sequentially, simultaneously co-administered, or administered prior to the composition of haNK cells and antibody as disclosed herein.

In another aspect of the inventive subject matter, disclosed herein is a kit, comprising a pharmaceutical composition, wherein the pharmaceutical composition comprise haNK cells, a therapeutic antibody, and optionally a cryopreservation medium. In this kit, the composition may be packaged in bags or vials suitable for storage in less than −85° C. Finally, the kit may also further comprising information, in electronic or paper form, comprising instructions for using the pharmaceutical composition.

Various objects, features, aspects, and advantages will become more apparent from the following detailed description of preferred embodiments, along with the accompanying drawing in which like numerals represent like components.

### BRIEF DESCRIPTION OF THE DRAWING

FIG. 1 depicts exemplary ADCC activity of haNK (with and without antibody preloaded) cells that were thawed, incubated on ice for 30 min, and tested.

FIG. 2A depicts exemplary ADCC activity of antibody preloaded haNK cells that were thawed and tested without post-thaw incubation.

FIG. 2B depicts exemplary ADCC activity of antibody preloaded haNK cells that were thawed, incubated on ice for 30 min, and tested.

FIG. 2C depicts exemplary ADCC activity of antibody preloaded haNK cells that were thawed, incubated on ice or room temperature for 30 min, and tested.

FIG. 3A depicts exemplary ADCC activity of antibody preloaded haNK cells that were thawed on ice for 30 min, followed by wash or no wash step, and tested.

FIG. 3B depicts exemplary ADCC activity of haNK cells that were thawed on ice for 30 min, followed by wash or no wash step, and tested in presence of exogenous Rituxan.

FIG. 4A depicts exemplary ADCC activity of antibody preloaded haNK cells that were prepared in different media, incubated on ice for 30 min, followed by wash or no wash step, and tested.

FIG. 4B depicts exemplary ADCC activity of haNK cells that were thawed on ice for 30 min, followed by wash or no wash step, and tested in the presence of exogenous Rituxan.

FIG. 4C depicts exemplary results in change of ADCC activity as a function of medium and wash step.

### DETAILED DESCRIPTION

Cell based therapies in the treatment of cancer are increasingly making use of various antibodies. Current therapeutic regimen consists of infusion of NK cells and monoclonal antibodies separately. However this leads to problems with dosing, and repeated bolus which in turn increases the cost of the treatment. The inventors have found a solution to this problem by making a pharmaceutical composition of haNK cells and antibody in a combined preparation. For example, the inventors made this composition by premixing therapeutic monoclonal antibodies such as Rituxan and haNK cells in 5% Human albumin, followed by mixing with CS-10 (1:1) and cryopreserved using controlled rate freezer and storage in vapor phase LN₂ freezer until ready to thaw and use as an infusion product. This premixed, combined preparation was shown to be effective and efficacious in killing cancer cells. Comparable killing activity was observed if the antibody was exogenously added to cryopreserved haNK cells in the assay.

Antibodies could be injected side by side with NK cells. However, in that case both are independent drugs and also the amount of antibodies used as a drug is higher. On the other hand, the advantage in the currently disclosed premixed, combined preparation is that the lower concentration of antibody together with cells is effective in killing. Moreover, haNK cells with antibody as combo would be used as off the shelf drug as one infusion rather than multiple injections.

Most monoclonal antibodies in oncology are administered in body-size-based dosing schedules. This partially controls the variability in both drug distribution and elimination between patients. However, dosing is a challenge in cell therapeutics where a combination of engineered cells and therapeutic antibodies are administered following different protocols. Further, repeated bolus increases the treatment cost. Hence combining both components in one dosing protocol is a desired solution.

The inventors have now discovered various compositions, methods and kits for pre-loading CD16⁺NK-92 (haNK) cells with antibodies, such as therapeutic antibodies. The compositions, methods and kits disclosed herein are contemplated to be a novel and more effective approach for tumor lysis. Preferably the composition comprising the haNK cells and the antibodies are in a form of a combined preparation, and the combined preparation is often cryopreserved until ready for use.

NK cells express Fc receptor, which can bind to the Fc portion of immunoglobulins, eliciting cell signals within NK cells. Once activated through Fc receptors by antibodies bound to tumor targets, NK cells are able to induce target lysis. This antibody-dependent cell-mediated cytotoxicity (ADCC) of target cells is employed for cancer treatment. haNK cells are NK-92 cells that are engineered to express high affinity CD16 receptors (FcγRIIIA) to facilitate ADCC mediated lysis of tumor cells. High affinity CD16 receptors can dock soluble IgG. However, until now, the binding strength/affinity as well as the ability of soluble antibodies to induce NK activation has not been tested. NK-92 cells are well known in the art (see e.g., Clin Cancer Res. 1998 November; 4(11):2859-68, and are also commercially available from NantKwest, San Diego, Calif.)

The inventors have now unexpectedly found that high affinity CD16 receptor on cryopreserved haNK cell may serve as a docking platform for soluble therapeutic antibodies and can induce ADCC when co-incubated with tumor target cells. Thus, the inventive concept disclosed herein relates to a pharmaceutical composition comprising a pharmaceutically acceptable carrier and therapeutically effective amounts of high affinity Natural Killer (haNK) cells and a therapeutic antibody in the form of a combined preparation.

Preferably the therapeutic antibody contemplated herein is one that causes necrosis or apoptosis of cancer cells. In one embodiment, the therapeutic antibody contemplated herein may be antibodies against TAA (tumor associated antigens), antibodies against cancer specific antigens, and antibodies against patient and tumor specific epitopes (neoepitopes). Optionally, or additionally, the therapeutic antibody contemplated herein may be antibodies against antigens found in necrotic cells and apoptotic cells.

Non-limiting examples of antibodies contemplated herein comprise Atezolizumab, Ofatumumab, Ipilimumab, Ramucirumab, Olaratumab, Elotuzumab, Necitumumab, Daratumumab, Dinutuximab, Avelumab, Durvalumab, Trastuzumab, Alemtuzumab, Bevacizumab, Pertuzumab, Obinutuzumab, Rituximab, and Cetuximab, and or various therapeutic or diagnostic antibodies with an IgG Fc portion. For example, suitable antibodies include (a) trastuzumab (Herceptin) which targets HER2 (ErbB2) by ADCC and inhibiting HER2 signaling, and is approved for the treatment of HER2-positive breast cancer, HER2-positive gastric or gastroesophageal junction carcinoma; (b) Bevacizumab (Avastin) which targets VEGF by inhibition of VEGF signaling, and is approved for the treatment of colorectal cancer, non-squamous non-small cell lung cancer, glioblastoma, or renal cell carcinoma; (c) Cetuximab (Erbitux) which targets EGFR (ErbB1) by ADCC and inhibition of EGFR signaling, and is approved for the treatment of squamous cell cancer of the head and neck (SCCHN); (d) Panitumumab (Vectibix) which targets EGFR (ErbB1) by inhibition of EGFR signaling, and is approved for the treatment of metastatic colorectal carcinoma; (e) Ipilimumab (Yervoy) which targets CTLA-4 by inhibition of CTLA-4 signaling, and is approved for the treatment of unresectable or metastatic melanoma; (f) Rituximab (Rituxan) which targets CD20 by ADCC, direct induction of apoptosis and CDC, and is approved for the treatment of CD20-positive B cell non-Hodgkin lymphoma (NHL) and chronic lymphocytic leukemia (CLL); (g) Alemtuzumab (Campath) which targets CD52 by direct induction of apoptosis and CDC, and is approved for the treatment of B cell CLL; (h) Ofatumumab (Arzerra) which targets CD20 by ADCC, and CDC, and is approved for the treatment of patients with CLL; (i) Gemtuzumab ozogamicin (Mylotarg) which targets CD33 by delivery of toxic payload, and is approved for the treatment of patients with CD33-positive acute myeloid leukemia; (l) ¹³¹I-Tositumomab (Bexxar) which targets CD20 by ADCC, induction of apoptosis and by delivery of the radio-isotope iodine-131, and is approved for the treatment of patients with CD20 antigen-expressing relapsed or refractory low grade, follicular, or transformed NHL. One skilled in the art will recognize that any other therapeutic antibody may also be used in the composition disclosed herein to practice of the present inventive concept.

Preferably, the haNK cell as disclosed herein is a NK-92 cell line derivative. It is further preferred that the haNK cells express recombinant IL2. Moreover, it is generally preferred that the haNK cell is genetically engineered to have a reduced expression of at least one inhibitory receptor and/or that the haNK cell is genetically engineered to express a CD16 158V variant. In some desirable embodiments, the haNK cell is genetically engineered to have a reduced expression of at least one inhibitory receptor.

Moreover, in further contemplated embodiments, the NK cells are irradiated before transfusion to prevent continuous cell division. While not limiting the inventive subject matter, the cells will typically be irradiated that abrogates cell division, but that still allows fast metabolic activity, and NK cell function, especially cytotoxic cell killing. Therefore, suitable radiation dosages for the NK cells are between 50 cGy and 2,000 cGy. In a preferred embodiment, the haNK cell may be irradiated before administration at a radiation dose of at least 500 cGy.

In some embodiments, the antibody is an anti-CD20 antibody (such as Rituximab, Ofatumumab, and/or ¹³¹I-Tositumomab), or an anti-CD16 antibody, such as MB311. The term “MB311” as used herein contemplates a fully humanized monoclonal antibody recognizing the tumor-associated antigen Lewis Y. This carbohydrate antigen is expressed on 60-90% of all epithelial cancers, with only limited expression on normal tissues, and thus represents an attractive target for cancer immunotherapy.

In some cases, the haNK cells and the antibody are chemically conjugated. The chemical conjugation may be done by any method known to a skilled chemist. Once preferred method of chemical conjugation is the Huisgen 1,3-dipolar cycloaddition reaction (“click chemistry”), as disclosed in H. C. Kolb; M. G. Finn; K. B. Sharpless (2001). “Click Chemistry: Diverse Chemical Function from a Few Good Reactions”. Angewandte Chemie International Edition. 40 (11): 2004-2021. The term “click chemistry” as used herein thus refers to such cycloaddition reactions and in particular to the cycloaddition of an azide and an alkyne—a reaction that may be in some embodiments be carried out under the catalysis of Cu(I) or under exposure to microwaves. Click reactions occur in one pot, are not disturbed by water, generate minimal and inoffensive byproducts, and are spring-loaded—characterized by a high thermodynamic driving force that drives it quickly and irreversibly to high yield of a single reaction product, with high reaction specificity (in some cases, with both regio- and stereo-specificity). These qualities make click reactions particularly suitable to the problem of isolating and targeting molecules in complex biological environments.

The pharmaceutical composition disclosed herein may further comprise a cryopreservation medium. The cryopreservation medium is contemplated to be cell-specific, optimized freeze media, which is designed to prepare and preserve cells in very low temperature environments (−70° C. to −120° C.). Furthermore, the cryopreservation medium is contemplated to provide a safe, protective environment during the freezing, storage, and thawing process for cells and tissues, and provide for enhanced cell viability and functionality while eliminating the need for serum, proteins or high levels of cytotoxic agents. In one preferred embodiment, the cryopreservation medium is CryoStor CS10. Furthermore, the cryopreservation medium is selected such that it favors antibody binding to haNK cells.

In another aspect of the inventive subject matter, the inventors have disclosed a method of treating a patient having cancer, comprising administering to the patient a combined preparation having therapeutically effective amounts of high affinity Natural Killer (haNK) cells and a therapeutic antibody. All kind of cancerous cells may be treated by using the compositions and methods disclosed herein. Thus, the methods disclosed herein are suitable for the treatment of Carcinoma, Sarcoma, Myeloma, Leukemia (liquid cancers or blood cancers), Lymphoma (solid cancers), or a mixed type of cancer such as adenosquamous carcinoma, mixed mesodermal tumor, carcinosarcoma, and teratorcarcinoma.

Preferably, the pharmaceutical compositions and methods disclosed herein may be formulated for delivery to a patient via any route of administration. “Route of administration” may refer to any administration pathway known in the art, including but not limited to aerosol, nasal, oral, transmucosal, transdermal or parenteral. Preferred routes of administration comprises inhalation, ocular administration, nasal instillation, parenteral administration, dermal administration, transdermal administration, buccal administration, rectal administration, sublingual administration, perilingual administration, nasal administration, topical administration or oral administration. “Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection. In some especially preferred embodiments, the composition may also be directly injected locally into the tumor (intratumoral administration).

Optionally, the method of treating cancer as disclosed herein may comprise a step of administering a further cancer treatment to the patient. The further cancer treatment comprises an immune therapy, chemotherapy, or radiotherapy. When immune therapy is the further cancer treatment, it comprises administration of a recombinant yeast or recombinant virus expressing a patient- and tumor-specific neoepitope. The chemotherapy may comprise administration of at least one of aldoxorubicin, cyclophosphamide, irinotecan, gemcitabine, capecitabine, 5-FU (5-fluorouracil), FOLFIRI (Folinic acid, fluorouracil and irinotecan), FOLFOX (Oxaliplatin, fluorouracil and leucovorin), and oxipiatin. The further cancer treatment may be administered separately, sequentially, simultaneously co-administered, or administered prior to the composition comprising haNK cells and therapeutic antibody as disclosed herein.

The pharmaceutical compositions of this disclosure are preferably delivered in a therapeutically effective amount. The precise therapeutically effective amount is that amount of the composition that will yield the most effective results in terms of efficacy of cancer treatment in a given subject. This amount may vary depending upon a variety of factors, including but not limited to the characteristics of the haNK cell, antibody, media compositions, etc, as well as the physiological condition of the subject (including age, sex, disease type and stage, general physical condition, responsiveness to a given dosage, and type of medication). One skilled in the clinical and pharmacological arts will be able to determine a therapeutically effective amount through routine experimentation, for instance, by monitoring a subject's response to administration of the pharmaceutical composition and adjusting the dosage accordingly. In a preferred embodiment, it is contemplated that the haNK cell is administered at a dosage of between 5×10⁵ cells/kg and 5×10⁸ cells/kg.

The composition disclosed herein may be made by a variety of techniques. In one preferred method, the haNK cells are mixed with a therapeutic antibody to making a combined preparation. The combined preparation is then frozen, followed by thawing prior to use. Preferably, haNK cells are in a media comprising about 10% albumin, or at least 8% albumin, or at least 6% albumin, or at least 5% albumin, or at least 3% albumin, or at least 1% albumin. The haNK cells and therapeutic antibody mixture is mixed with a cryopreservation medium. The ratio of the combined preparation and the cryopreservation medium may be at least 3:1, or at least 2:1, or at least 1:1, or at least 1:2, or at least 1:3.

The composition is frozen to a temperature between −50° C. to −100° C. Once the cells have reached a temperature between −50° C. to −100° C., they are cooled even further by putting the cells in liquid nitrogen, say to a temperature than −120° C. The combined preparation is frozen until it is ready for use, or in some cases, at least 1 month, or at least 2 weeks, or at least 1 week, or at least 5 days, or at least 3 days, or at least one day, or at least 12 hours, or at least 6 hours, or at least 2 hours, or at least 1 hour. In some preferred embodiments, the frozen combined preparation is irradiated with a fixed dose of X-ray irradiation to impair the proliferative ability of the haNK cells. After the thawing step, the combined preparation is incubated on ice or room temperature. The thawing step may be in ice or at room temperature, or any other temperature between 0° C. to 100° C., or more preferably between 0° C. to 40° C., or even more preferably between 0° C. to 25° C. Once the composition has thawed, it is allowed to incubate in ice or room temperature (between 0° C. to 25° C.) for at least 5 hours, or more preferably at least 4 hours, or at least 3 hours, or at least 2 hours, or at least 1 hour, or at least 30 minutes, or at least 15 minutes, or at least 10 minutes.

In another aspect of this disclosure, the inventive concept is directed to preparation of and use of a kit comprising a pharmaceutical composition of haNK cells and a therapeutic antibody. Optionally, the pharmaceutical composition in the kit may also comprise a cryopreservation medium. The kit is useful for practicing the inventive method of treating cancer or tumors. The kit is an assemblage of materials or components, including at least one of the inventive compositions as described throughout this disclosure.

The exact nature of the components configured in the inventive kit depends on its intended purpose. For example, some embodiments are configured for the purpose of treating a tumor and/or cancer. In that case, the antibody used is specific to the cancer, such as trastuzumab for breast cancer. Moreover, the composition may further comprise a pharmaceutically acceptable carrier that favors the binding of the trastuzumab to the haNK cells.

In one embodiment, the kit is configured particularly for the purpose of treating mammalian subjects. In another embodiment, the kit is configured particularly for the purpose of treating human subjects. In further embodiments, the kit is configured for veterinary applications, treating subjects such as, but not limited to, farm animals, domestic animals, and laboratory animals.

Instructions for use may be included in the kit. “Instructions for use” typically include a tangible expression describing the technique to be employed in using the components of the kit to effect a desired outcome, such as to decrease or kill a tumor. Optionally, the kit also contains other useful components, such as, diluents, buffers, pharmaceutically acceptable carriers, syringes, catheters, applicators, pipetting or measuring tools, bandaging materials or other useful paraphernalia as will be readily recognized by those of skill in the art.

The materials or components assembled in the kit can be provided to the practitioner stored in any convenient and suitable ways that preserve their operability and utility. For example, as contemplated herein, the components are more preferably provided in a frozen temperatures, typically less than −85° C. The components are typically contained in suitable packaging material(s). As employed herein, the phrase “packaging material” refers to one or more physical structures used to house the contents of the kit, such as inventive compositions and the like. The packaging material is constructed by well-known methods, preferably to provide a sterile, contaminant-free environment. As used herein, the term “package” refers to a suitable solid matrix or material such as glass, plastic, paper, foil, and the like, capable of holding the individual kit components. The packaging material generally has an external label which indicates the contents and/or purpose of the kit and/or its components.

Embodiments of the present disclosure are further described in the following examples. The examples are merely illustrative and do not in any way limit the scope of the invention as claimed.

### EXAMPLES

### Example 1

In one exemplary embodiment, the inventors mixed haNK cells (commercially available from NantKwest, San Diego) in 5% Albumin (human) with an anti-CD20 antibody (Rituxan). Subsequently, an equivalent volume (1:1) of CryoStor10 (CS10) was added and the mixture was transferred into CellFreeze infusion bags and vials. haNK cells without antibodies were also included in the study as a negative control. The filled infusion bags and vials were subsequently cryopreserved to ≤−85° C. using a controlled rate freezer. The cryopreserved product was then transferred to liquid nitrogen vapor phase (≤−120° C.) freezer for storage. The frozen products were irradiated with a fixed dose of X-ray irradiation to impair the proliferative ability of the haNK cells. Upon thaw, cells were incubated with Calcein labeled Ramos (target) cells expressing CD20 and lysis was evaluated using calcein release assay.

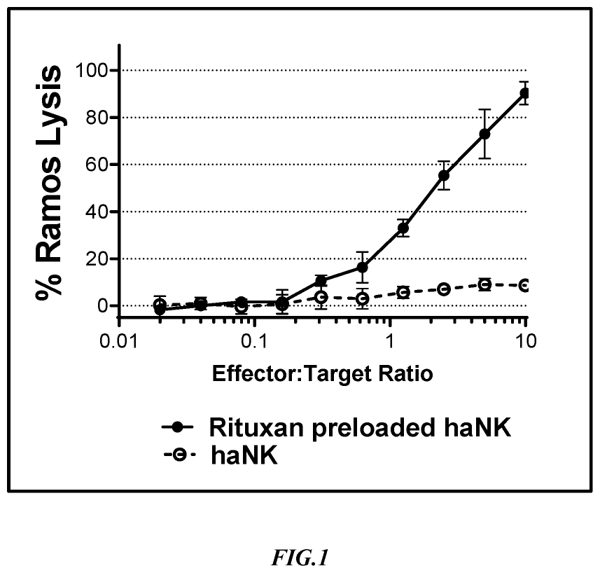

Potent Ramos lysis was induced by Rituxan pre-loaded haNK cells but not by haNK cells alone as shown in FIG. 1, which illustrates ADCC activity of the haNK cells after thaw. Here, haNK cells were either pre-loaded with Rituxan antibody at 1 μg/mL, or not preloaded and then cryopreserved. The cryopreserved cells were then irradiated with 15 Gy using RS-2000 X-ray irradiator. Cells were thawed and incubated on ice for 30 minutes, and directly added to the assay plate. Percentage Ramos lysis was evaluated using a standard calcein release assay.

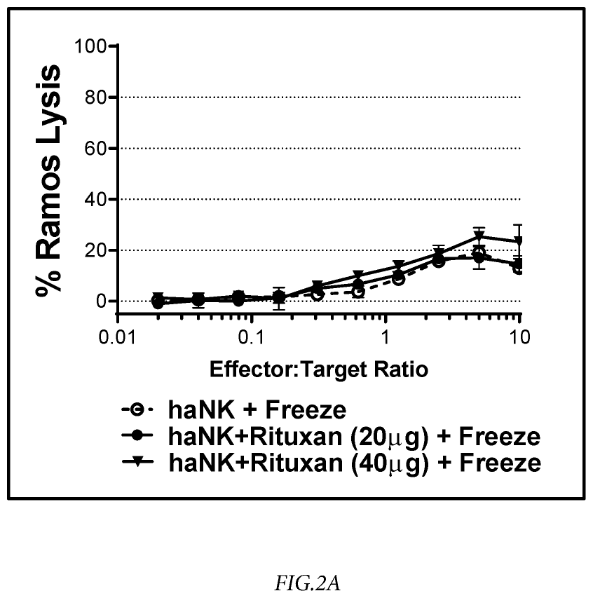

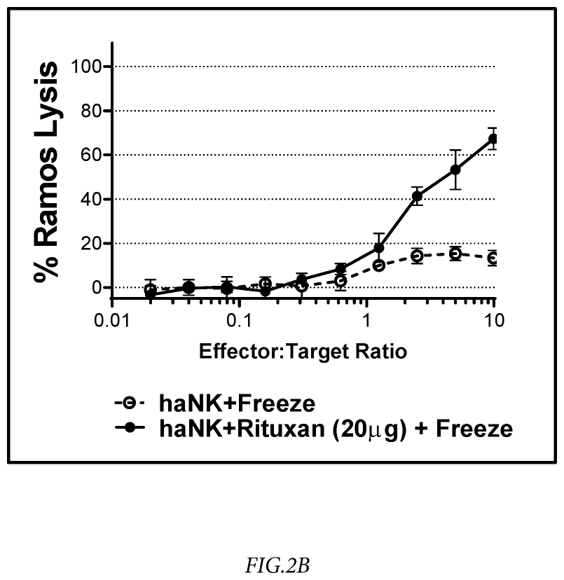

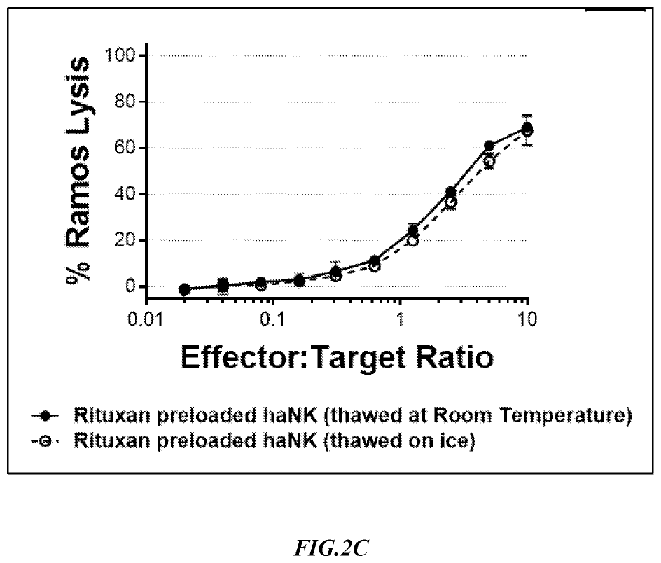

In further examples, the inventors investigated the timing of ADCC upon thaw. Interestingly, the inventors discovered that a post-thaw hold time of 10-30 minutes (or more in some instances) on ice or room temperature had a significant impact on the potency of ADCC function of the haNK cells. For example, haNK cells were mixed or not with Rituxan antibody at 1-2 μg/mL and cryopreserved. Cryopreserved cells were irradiated with 15 Gy using RS-2000 X-ray irradiator. Thawing was then conducted at different protocols as follows: FIG. 2A shows the results for cells that were thawed, washed, and tested without any post-thaw incubation time and substantially no ADCC based cell killing was observed. In contrast, when the cells were thawed and incubated on ice (FIG. 2B) or at room temperature (RT) or ice (FIG. 2C) for 30 minutes, substantially improved ADCC cytotoxicity was observed. Such activity was also observed after a wash step that was intended to remove unbound Rituximab. Once more, in FIGS. 2A-2C % Ramos lysis was evaluated using a standard calcein release assay.

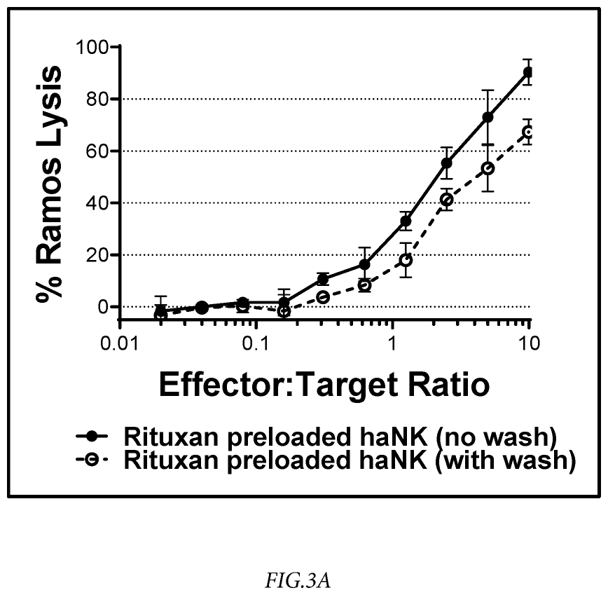

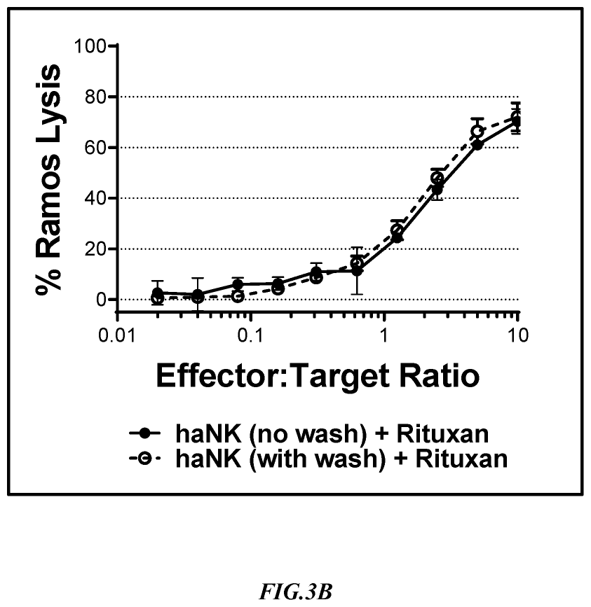

In yet another example, the inventors also evaluated the contribution of free antibodies in the thawed haNK product. To that end, the antibody preloaded cells were washed post thaw once by centrifugation at 336×g (1200 RPM) for 5 minutes followed by an ADCC assay. As illustrated in FIGS. 3A-3B, haNK cells were mixed or not with Rituxan antibody at 1 μg/mL and cryopreserved. Cryopreserved cells were irradiated with 15 Gy using RS-2000 X-ray irradiator. FIG. 3A depicts the results for ADCC upon thaw. Here, Rituxan pre-loaded haNK cells were either washed or directly added to the assay plate without wash step. To compare, upon thaw, haNK cells (not antibody preloaded) were either washed or directly added to the assay plate containing Rituxan bound Ramos cells and the results are shown in FIG. 3B. As can be readily seen, the wash step did not affect the cells per se when they were not preloaded with antibody.

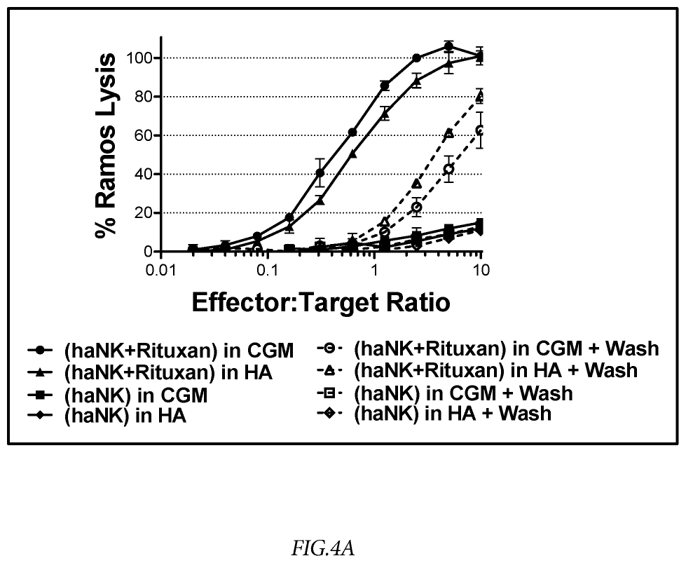

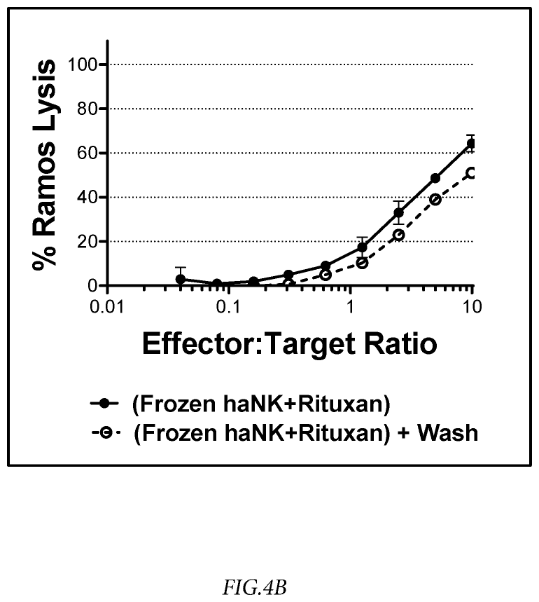

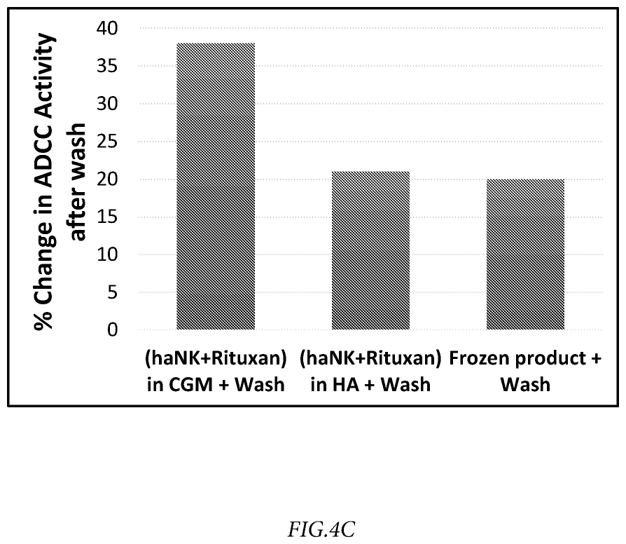

In still further experiments, to evaluate the contribution of components of the freezing media in the Rituxan preloaded haNK product, cells were tested to induce ADCC before freezing. Fresh haNK cells suspended in complete growth media (CGM) or Albumin (Human) were mixed or not with Rituxan antibody at 1 μg/mL FIG. 4A shows activity of fresh product with or without wash step. Similarly, FIG. 4B shows results upon thaw where haNK cells were either washed or directly added to the assay plate. FIG. 4C shows the reduced % change in ADCC activity when cells were suspended in Albumin (HA) vs complete growth media. Interestingly, these result indicate that the type of media (here: complete growth media and human albumin media) beneficially contributed to better binding of antibodies to haNK cells.

### Example 2

In further examples, the inventors developed a new technology based on the premise that tumor cells have aberrant carbohydrate glycosylation and that those structures can be used to specifically target mAbs, i.e the Lewis system antigens. In this method, mAbs with increased FcR affinity are generated using the moss expression system. There are currently several technologies that focus on making mAb with modified FcR that bind with higher affinity to NK cells, and suitable technologies to be used herein are those that are safe and effective in humans.

Preferably, a mAb product is combined with a systemic CD-16 expressing NK-92 cell infusion (dual therapy). A noteworthy point is that a portion of the NK cell Fc receptors would be occupied by human serum IgG before the therapeutic mAb could bind the NK cell. Pre-binding NK cells with MB311 before administration could be a solution. Such a construct would likely be large enough to become lodged in the pulmonary capillary bed when administered in a peripheral vein. Something similar would be expected if administered by an arterial route. MB311 is a fully humanized monoclonal antibody recognizing the tumor-associated antigen Lewis Y.

In another view, the pre-bound complex of NK92 with its CD16 receptors saturated with MB311 or other similar mAb, may have the limitation of easy dissociation and recombining with plasma IgG. Generally, the Fc affinity is not very high, even on the 176V expressing NK92 cells. The antibody would then become free to possibly elicit toxicity. This may be in part addressed by utilizing MB311 grown in the moss reactor because the Ab has a better chance of staying bound (up to 40 fold greater affinity for Fc than MB311).

Routes of administration may be localized administration (pleural/abdominal effusions of metastatic cancer) or local injection into tumor. In case of local injection, tumor penetration may be a concern, which can be addressed by inclusion of collagenase/proteinase to mechanically break up a ‘hard’ tumor for improved penetration/access.

Blood group related antigens represent a group of carbohydrate determinants carried on both glycolipids and glycoproteins. They are usually mucin-type, and are detected on erythrocytes, certain epithelial cells, and in secretions of certain individuals. Sixteen genetically and biosynthetically distinct but inter-related specificities belong to this group of antigens, including A, B, H, Lewis a, Lewis b, Lewis x, Lewis y, and precursor type 1 chain antigens. Lewis y (type 2 chain) antigen is a difucosylated tetrasaccharide found on the Type 2 blood group oligosaccharides of glycolipids and glycoproteins. It is expressed in large bowel tumors and colorectal carcinomas. The Lewis y antigen may also act as a clinical marker for the diagnosis and prognosis of cholangiocarcinoma, hepatocellular carcinoma and breast cancer.

### Example 3

In one embodiment, a phase I/II, open label trial of Lewis Y specific monoclonal antibody IGN311 evaluated the safety and efficacy in patients with malignant effusion. In brief, treatment of CRC patients with the humanized mAb IGN311 targeting the carbohydrate Lewis Y eliminated circulating tumor cells in blood and thereby confirmed the clinical profile of the parent murine antibody ABL364, which showed elimination of Lewis Y and cytokeratin positive cells in bone marrow of pts with breast cancer.

An open-label, single treatment arm, uncontrolled study with IGN311 (100 mg per dose, intravenously on day 1 and 7) in patients with malignant effusion (ascites or pleural effusion) is being conducted with the primary objective to examine safety and tolerability. Secondary objectives are volumetric measurement of the malignant effusion and to obtain data for several immunological parameters.

4 patients (2 patients with gastric cancer and malignant ascites, 2 patients with breast cancer and malignant pleural effusion/ascites) have completed the study until December 2005. IGN311 was well tolerated with only one patient showing up to grade 2 nausea, vomiting and skin rashes as side effect after 1^{st} application which was easily managed. In all pts significant levels of IGN311 were measured followed by an increase in CD45 positive cells in the effusion. The patient with the highest level of Lewis Y expressing tumor cells showed a reduction of effusion volume during treatment.

Thus, these experiments showed that IGN311 was well tolerated, permeated into malignant effusion and attracted immune cells leading to decreased tumor cell counts in the effusion. In the case of strong Lewis Y expression of malignant cells in the effusion a reduction of the effusion volume could be demonstrated.

As used herein, the term “administering” a pharmaceutical composition or drug refers to both direct and indirect administration of the pharmaceutical composition or drug, wherein direct administration of the pharmaceutical composition or drug is typically performed by a health care professional (e.g., physician, nurse, etc.), and wherein indirect administration includes a step of providing or making available the pharmaceutical composition or drug to the health care professional for direct administration (e.g., via injection, infusion, oral delivery, topical delivery, etc.). Most preferably, the cells or exosomes are administered via subcutaneous or subdermal injection. However, in other contemplated aspects, administration may also be intravenous injection. Alternatively, or additionally, antigen presenting cells may be isolated or grown from cells of the patient, infected in vitro, and then transfused to the patient. Therefore, it should be appreciated that contemplated systems and methods can be considered a complete drug discovery system (e.g., drug discovery, treatment protocol, validation, etc.) for highly personalized cancer treatment.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the the full scope of the present disclosure, and does not pose a limitation on the scope of the invention otherwise claimed. No language in the specification should be construed as indicating any non-claimed element essential to the practice of the claimed invention.

It should be apparent to those skilled in the art that many more modifications besides those already described are possible without departing from the full scope of the concepts disclosed herein. The disclosed subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

1. A pharmaceutical composition comprising a pharmaceutically acceptable carrier and therapeutically effective amounts of high affinity Natural Killer (haNK) cells and a therapeutic antibody in the form of a combined preparation.

2. The pharmaceutical composition of claim 1, wherein the haNK cell is a NK-92 cell line derivative.

3. The pharmaceutical composition of claim 1 wherein the haNK cell further expresses recombinant IL2.

4. The pharmaceutical composition of claim 1 wherein the haNK cell is genetically engineered to have a reduced expression of at least one inhibitory receptor.

5. The pharmaceutical composition of claim 1 wherein the haNK cell is irradiated before administration at a radiation dose of at least 500 cGy.

6. The pharmaceutical composition of claim 1 wherein the antibody is an anti-CD20 antibody.

7. The pharmaceutical composition of claim 6, wherein the anti-CD20 antibody is Rituximab.

8. The pharmaceutical composition of claim 1 wherein the antibody is selected from the group consisting of Atezolizumab, Ofatumumab, Ipilimumab, Ramucirumab, Olaratumab, Elotuzumab, Necitumumab, Daratumumab, Dinutuximab, Avelumab, Durvalumab, Trastuzumab, Alemtuzumab, Bevacizumab, Pertuzumab, Obinutuzumab, Rituximab, and Cetuximab.

9. The pharmaceutical composition of claim 1 further comprising a cryopreservation medium.

10. The pharmaceutical composition of claim 9, wherein the cryopreservation medium is CryoStor CS10.

11. The pharmaceutical composition of claim 9, wherein the cryopreservation medium increases or stabilizes antibody binding to the haNK cells.

12. A method of making a composition according to claim 1, comprising:
mixing haNK cells with a therapeutic antibody to produce a combined preparation;
freezing the combined preparation; and
thawing the combined preparation.

13. The method of claim 12, wherein the haNK cells are in a medium comprising about 5% albumin.

14. The method of claim 12, wherein the haNK cells and therapeutic antibody mixture is mixed with an equivalent volume of a cryopreservation medium.

15. The method of claim 12, wherein the combined preparation is frozen to a temperature less than −80° C.

16. The method of claim 12, wherein the combined preparation is frozen to a temperature less than −120° C.

17. The method of claim 12, wherein the frozen combined preparation is irradiated with a fixed dose of X-ray irradiation to impair a proliferative ability of the haNK cells.

18. The method of claim 12, wherein, after the thawing step, the combined preparation is incubated on ice or room temperature for at least 10 minutes.

19-31. (canceled)

32. A kit comprising a pharmaceutical composition, wherein the pharmaceutical composition comprises haNK cells, a therapeutic antibody, and a cryopreservation medium or a cell growth medium.

33. The kit of claim 32, wherein the composition is packaged in bags or vials suitable for storage in less than −85° C.

34. (canceled)
